(19) United States
(12) Patent Application Publication
(10) Pub. No.: US 2026/0024637 A1
(43) Pub. Date: Jan. 22, 2026

(54) AI Clinical Engine: System for Aggregating Medical Docs to Optimize Consult Preparation and Findings

(71) Applicant: Fuse Oncology, Boone, NC (US)

(72) Inventors: Yusuf Fawzy ELNADY, Blacksburg, VA (US); Matthew Richard TERRY, Jonesboro, AR (US); David Bryan UNDERWOOD, Youngsville, NC (US); David Brian WIANT, Kernersville, NC (US); George James BAULER, II, Boone, NC (US); Lauren Kaylie MANCUSO, Durham, NC (US); Christel Johanna SMITH, Santa Barbara, CA (US); Benjamin Jeremiah SINTAY, Summerfield, NC (US)

(21) Appl. No.: 19/273,842

(22) Filed: Jul. 18, 2025

Related U.S. Application Data
(60) Provisional application No. 63/673,662, filed on Jul. 19, 2024.

(51) Int. Cl. G16H 15/00; G06F 40/10; G06V 30/14; G16H 10/60

(52) U.S. Cl. CPC G16H 15/00; G06F 40/10; G06V 30/14; G16H 10/60

(57) ABSTRACT

A system and method are disclosed for generating a History of Present Illness (HPI) summary by extracting and synthesizing clinical data from unstructured or semi-structured medical documents. The invention utilizes natural language processing (NLP) and optical character recognition (OCR), where applicable, to identify relevant patient information such as symptom onset, duration, location, and progression. A disease-specific workflow determines the clinical relevance of extracted elements, which are then compiled into a structured or narrative HPI summary. The system further includes confidence scoring, physician validation interfaces, and dynamic regeneration of the HPI based on user corrections. Additional components include disease-aware document retrieval, timeline-based visualization of patient events, and AI-generated sections of a consultation note beyond the HPI. This invention enhances clinical efficiency, accuracy, and interoperability by automating and contextualizing the HPI generation process for use in electronic health record (EHR) systems.

### CROSS-REFERENCE TO REPLATED APPLICATIONS

This application claims the benefit of U.S. Provisional Patent Application No. 63/673,662 filed Jul. 19, 2024, which is incorporated herein in its entirety.

### FIELD OF THE INVENTION

The History of Present Illness (HPI) Generator invention streamlines the process of gathering and documenting essential patient information by utilizing various technologies to automate the extraction and structuring of HPI data. It then provides crucial information regarding the patient's symptoms, onset, duration, and progression necessary for accurate diagnosis of the type and stage of cancer and other diseases.

### BACKGROUND OF THE INVENTION

The history of present illness (HPI) is a detailed account of the patient's current symptoms and the circumstances surrounding them. It's a crucial part of every patient's medical history by providing context for the patient's illness and helping clinicians understand the nature, progression, and treatment of the illness. In radiation oncology, a detailed and accurate HPI is essential. It directly informs the diagnosis, staging, and development of individualized treatment plans for cancer patients. Moreover, the completeness and precision of the HPI significantly influence downstream decisions regarding imaging, pathology, therapeutic modalities, and patient monitoring strategies

However, the process of generating a History of Present Illness (HPI) is a critical but time-consuming component of medical documentation, particularly within the field of radiation oncology. The HPI includes vital information such as the patient's symptoms, their onset, duration, and progression. Traditionally, compiling this information requires significant manual effort and clinical expertise, often taking a physician or trained nurse valuable time in their busy workday to render for each and every patient they treat or diagnose. Moreover, this process must be repeated for every new consultation and represents a substantial administrative burden on already overextended clinical staff resources.

Despite the increasing adoption of electronic health records (EHRs) and other digital documentation tools, no existing system provides a robust, scalable, and clinically safe solution for automating HPI generation using unstructured or semi-structured medical data. Many available tools either lack the ability to ingest multiple data formats (e.g., PDF, DOCX, scanned documents) or fail to apply advanced language models for nuanced clinical interpretation. Furthermore, few, if any, tools integrate safe de-identification mechanisms to facilitate external AI processing while protecting patient health information (PHI).

Unfortunately the current practice of costly, tedious, time-consuming and potentially error prone human labor is still relegated to generating structured HPI summaries from diverse medical data sources while maintaining clinical relevance, accuracy, and data privacy.

### BRIEF SUMMARY OF THE INVENTION

Embodiments of the present invention pertain to an automated system and method for automatically generating a structured and clinically accurate History of Present Illness (HPI) using artificial intelligence and specialized data workflows. The system ingests unstructured and semi-structured medical documents (e.g., PDFs, DOCX files, scanned records, and EHR exports) and processes them using optical character recognition (OCR), natural language processing (NLP), and large language models (LLMs) to extract relevant clinical information. Tailored workflows for specific disease types, such as prostate, breast, or lung cancer, enable the system to identify and prioritize key documents and data elements for each clinical context. The invention produces a coherent, well-structured HPI paragraph and/or a set of structured data fields, reducing the time and effort required from clinicians while improving consistency and accuracy. The following detailed description together with the accompanying drawings will provide a better understanding of the nature and advantages of the present invention.

### BRIEF DESCRIPTION OF THE DRAWINGS

Embodiments of the present invention are illustrated by way of example, and not by way of limitation, in the figures of the accompanying drawings and in which like reference numerals refer to similar elements.

FIG. 1 shows an overview of one embodiment of the present invention.

FIG. 2 shows a flowchart of the Queuing and Basic Process Flow.

FIG. 3 shows a block diagram for a system and methodology for generating a unified patient timeline view.

FIG. 4 shows an exemplary FuseDocs listing all scheduled notes.

FIG. 5 shows a two-panel view after clicking on the consult note.

FIG. 6 shows a UI display window or panel after clicking the “Verify Information” button.

FIG. 7 shows a display of an example of the configurations that tracks each disease site.

FIG. 8 shows an exemplary Consult Note AI workflow diagram.

### DETAILED DESCRIPTION OF THE INVENTION

Reference will now be made in detail to embodiments, examples of which are illustrated in the accompanying drawings. While the embodiments will be described in conjunction with the drawings, it will be understood that they are not intended to limit the embodiments. On the contrary, the embodiments are intended to cover alternatives, modifications and equivalents. Furthermore, in the following detailed description, numerous specific details are set forth in order to provide a thorough understanding. However, it will be recognized by one of ordinary skill in the art that the embodiments may be practiced without these specific details. In other instances, well-known methods, procedures, components, and circuits have not been described in detail as not to unnecessarily obscure aspects of the embodiments.

The present invention provides a system and method for the automated generation of a History of Present Illness (HPI) summary using artificial intelligence (AI), natural language processing (NLP), and clinical workflow automation. The system is designed to transform raw clinical data from a variety of formats into a structured or narrative HPI suitable for use by clinicians, particularly in fields such as radiation oncology where timely and accurate clinical histories are critical for treatment planning. Basically, the invention streamlines the traditionally manual process of collecting and composing HPI narratives by leveraging AI technologies to extract relevant data elements and generate medically coherent summaries. Through the utilization of artificial intelligence and specialized work flows, patient care in the field of radiation oncology will benefit significantly due to enhanced efficiency and accuracy of HPI generation. The HPI Generator can curate relevant documents for types of cancer and intelligently identifies the essential information using disease specific specialized work flows and artificial intelligence.

In operation, the system receives medical documentation from multiple input sources, including but not limited to electronic health records (EHRs), clinician notes, pathology reports, radiology findings, and lab results. These documents may be structured, semi-structured, or unstructured, and may be provided in formats such as DOCX, PDF, HL7, FHIR, or image-based files such as scanned intake forms. A document ingestion module processes these inputs and, when necessary, passes image-based content to an optical character recognition (OCR) engine that extracts machine-readable text.

In one embodiment, the system includes an anonymization module that detects and removes protected health information (PHI) from the ingested documents prior to further processing. The anonymization process may involve rule-based masking, token replacement, or NLP-based named entity recognition to identify patient names, dates of birth, medical record numbers, addresses, and other identifiers. These tokens may be stored in a mapping table that enables later re-identification once AI processing is complete. This embodiment is particularly useful when the system interacts with external AI services or when privacy-preserving computation is desired in cloud or third-party processing environments. However, in other embodiments where data remains within a secure, trusted environment (e.g., a hospital's internal network or HIPAA-compliant private server) the anonymization module may be omitted or bypassed entirely.

Once text data is available, whether anonymized or not, the system applies a configurable clinical workflow engine that selects a disease-specific pipeline based on input parameters or document content. For example, a prostate cancer workflow may prioritize PSA results, urology notes, and prostate MRI reports, whereas a breast cancer workflow may focus on mammogram findings and biopsy results. The workflow defines which document types and data elements are relevant and applies logic to guide the AI extraction process. It should be noted that critical clinical information remains readily accessible and workflows align with real-world usage patterns in oncology settings. Notably, embodiments of the present invention support not just the consulting physician, but the broader care team, including nurses, patient navigators, and even the patients themselves. This improves information access and reducing documentation burden.

The selected and preprocessed documents are then analyzed by a machine learning engine comprising one or more large language models (LLMs), such as transformer-based architectures fine-tuned on medical corpora. These models extract structured clinical information relevant to the HPI, including symptom onset, duration, location, associated features, modifying factors, past interventions, and comorbidities. In some embodiments, the system may interface with a vector store or retrieval-augmented generation (RAG) framework that queries a corpus of previously processed HPIs or clinical documents. Retrieved content may be used to provide contextual signals to the model or to offer example outputs that guide the current generation task.

The extracted information is passed to an HPI composition engine that synthesizes one or more candidate summaries. These may be formatted as free-text narratives appropriate for physician review or as structured data suitable for downstream clinical applications. Each summary may be annotated with metadata such as confidence scores, data provenance links, or field-level validations. In embodiments using anonymization, a de-anonymization module reverses the earlier masking process, restoring original PHI tokens to create a clinically usable, patient-specific document.

The completed HPI summary is then routed to an output module that renders the final output in the desired format, including PDF, plaintext, HL7, FHIR JSON, or CDA-compatible formats for integration with EHR systems. The system may support export via secure web interfaces, APIs, or direct embedding into clinician workflows. Logging and audit mechanisms ensure that all steps are recorded in secure storage for compliance, quality control, and traceability.

By integrating clinical domain logic, AI-based document interpretation, and optionally privacy-preserving preprocessing, the present invention enables the efficient, accurate, and scalable generation of HPIs. The inclusion of anonymization as an optional embodiment allows the system to flexibly adapt to different deployment contexts, including cloud-hosted, on-premise, and hybrid configurations, thereby enhancing usability and regulatory compliance across a broad range of healthcare environments. Thereby, the HPI Generator expedites the process using artificial intelligence and specialized work flows to improve the extraction and writing of HPIs from various data sources, file formats, and data lakes and provides a well-structured, comprehensive paragraph with all the necessary information. Other key benefits of the HPI Generator are the pathways determining relevant information needed, identification to the physician that there is missing information, summarization of patient history, and the citation or reference of what documents specific information came from. The HPI Generator improves patient care through the use of AI and specialized workflows. While these benefits are shown through the increased efficiency and accuracy of HPI generation in the field of radiation oncology, in other embodiments, the tool can be utilized for other areas, such as the writing and filing of more sections of the initial consultation note, including Family History, Allergies/Alerts, Current Medications, Lab Results, Pathology, and Radiology.

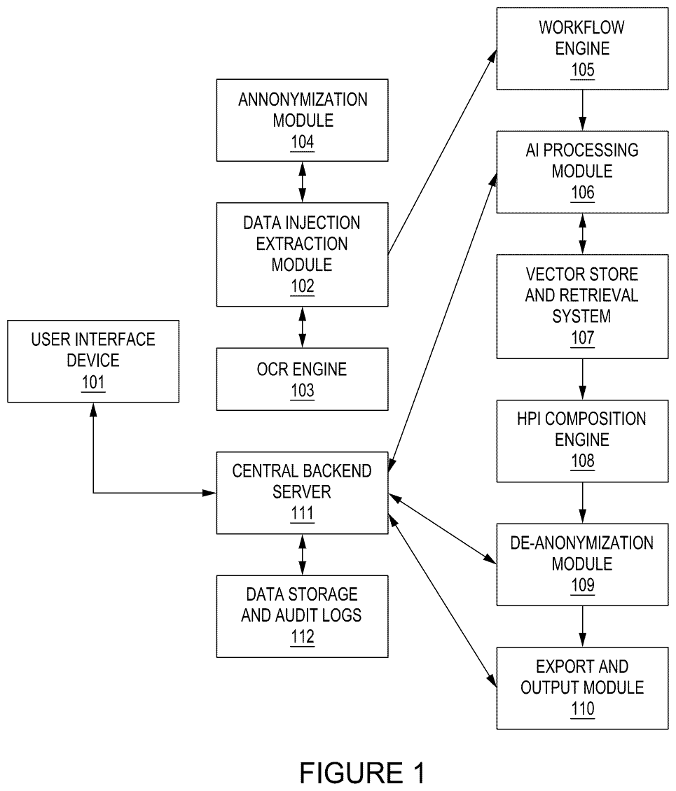

Referring now to FIG. 1, a block diagram of an exemplary system architecture for the History of Present Illness (HPI) Generator is shown. The system 100 may be implemented using one or more computing devices, such as servers, workstations, or cloud-based infrastructure. A user interacts with the system through a user interface device 101, which may be a desktop computer, tablet, or terminal that allows clinicians to upload medical documents, configure disease-specific workflows, and review or edit the generated HPI output.

The system includes a data ingestion and extraction module 102 configured to retrieve and receive clinical documents from various sources and in multiple formats, including PDF, DOCX, image files, structured HL7/FHIR messages, or free-text exports from electronic health records (EHRs). In one embodiment, document retrieval is driven by a dynamic, disease-specific search methodology that compensates for the inconsistent labeling practices seen across healthcare institutions. Hospitals frequently name documents differently, making standardized retrieval difficult. To overcome this, one embodiment of the system of the present invention utilizes an oncology-informed keyword and document-type search framework. For each cancer type, a tailored configuration file specifies which documents are clinically relevant (e.g., mammograms for breast cancer or PSA values for prostate cancer). These configurations are constantly updated to align with global disease prevalence trends and ensure that the most relevant and necessary records are retrieved for each consult. When documents are scanned or image-based, they are processed by an optical character recognition (OCR) engine 103, which performs image pre-processing such as de-skewing and noise removal and extracts machine-readable text from the image. The data injection extraction module is designed to manage a broad spectrum of medical document formats, including non-digital inputs such as scanned, faxed, or handwritten records. The specialized OCR engine 103, fine-tuned for the healthcare domain, is capable of extracting critical information from poor-quality inputs, recognizing handwritten annotations, circled values, or marginal notes that standard extraction tools overlook. Each document is processed individually, and essential patient information, referred to as “keys” is extracted and structured. These keys may include values such as lab results, diagnosis dates, pathology findings, and other clinical markers essential for oncology workflows.

Following extraction, the system aggregates all keys into a unified, structured data layer, ensuring that each data point retains a citation linking back to its source document. This feature supports clinical transparency and physician trust. Physicians can interact with the structured data layer, correcting any inaccuracies or ambiguous values directly through the interface. When edits are made, the History of Present Illness (HPI) and other relevant documentation sections are automatically regenerated to reflect these updates. Planned features include assigning confidence scores to each data point, which would alert physicians to values derived from lower-quality documents or ambiguous extractions (e.g., visual confusion between a “6” and an “8”).

In one embodiment, to ensure privacy compliance, the extracted data is passed to an anonymization module 104, which uses rules-based algorithms and natural language processing to detect and redact or tokenize protected health information (PHI), such as patient names, dates of birth, and medical record numbers. This step enables downstream AI processing without risk of exposing sensitive patient identifiers.

Next, the preprocessed documents are routed to a workflow engine 105, which identifies and initiates the appropriate disease-specific workflow. For instance, in a prostate cancer case, the system may prioritize documents such as PSA lab reports, urologist notes, and prostate MRIs. The workflow engine determines which documents and data elements are relevant for that particular condition and routes them accordingly. The selected documents are then processed by an AI processing module 106, which employs large language models (LLMs), such as transformer-based architectures, possibly in conjunction with external services and libraries, such as LangChain, Vector Stores, OpenAI, Microsoft Azure, docx2python, transformers, pdfplumber, and Flask. While these are some integrated scripts, it will be understood that they are not intended to limit the invention to the use of only those listed herein. These models extract relevant clinical elements such as symptom onset, duration, associated factors, and progression, which are core to a high-quality HPI.

In one embodiment, the AI processing module 106 leverages a curated library of disease-specific templates. These templates function like structured “Mad Libs,” with placeholders mapped to key clinical concepts. They are drawn from evidence-based oncology guidelines and formatted in styles tailored to individual physician preferences. They can range from narrative prose to bullet points. This approach ensures a high degree of control over the AI output, eliminating the risks associated with unstructured or purely generative text generation. The same keys used in the HPI generation are also reused across other portions of the consultation note, such as the review of systems, pathology summary, lab report, and imaging review. Thereby documentation and consistency are improved.

In one embodiment, the AI module or engine is hosted in a secure, cloud-based environment (e.g., Microsoft Azure), ensuring HIPAA-compliant storage, compute scalability, and integration with hospital IT infrastructure. Unlike generic AI summarization tools or record retrieval systems, various embodiments of the present invention integrate domain-specific knowledge, structured templates, citation-backed outputs, and interactive timelines to deliver a comprehensive oncology workflow solution.

In conjunction with the AI module, the system includes a vector store and retrieval system 107, which enables semantic search and case similarity matching. This allows the system to draw upon past processed cases and pre-indexed clinical documents to enrich the quality and context of the generated HPI. The extracted information is forwarded to an HPI composition engine 108, which synthesizes the data into one or more well-structured HPI summaries. These may be rendered as narrative paragraphs or as structured data fields, with each version optionally annotated with confidence scores or citations linking back to the source content. Once the HPI is composed, it is passed to a de-anonymization module 109, which securely re-inserts the original PHI by mapping tokens back to their corresponding patient identifiers. This ensures that the final output is complete and suitable for clinical use.

The finalized HPI is then routed to an output formatter and export module 110, which renders the summary in a format appropriate for review, download, or integration into the user's clinical system. Supported output formats may include HL7, FHIR-compliant JSON, PDF, or simple text. The physician or end-user is able to choose the best format for their needs as the HPI Generator provides multiple suggestions, giving users flexibility. Key elements are also able to be extracted as categorical data for the end user, providing easy comparison and verification of the data to ensure accuracy. All processes are coordinated by a central backend server 111, which manages inter-module communication, user authentication, access control, task queuing, and system logging. This backend may reside in a HIPAA-compliant cloud environment or on a private on-premises server, depending on deployment requirements. Lastly, the system includes secure data storage and audit logs 112, where all uploaded files, intermediate data states, user actions, model outputs, and logs are stored for traceability, compliance, and performance auditing.

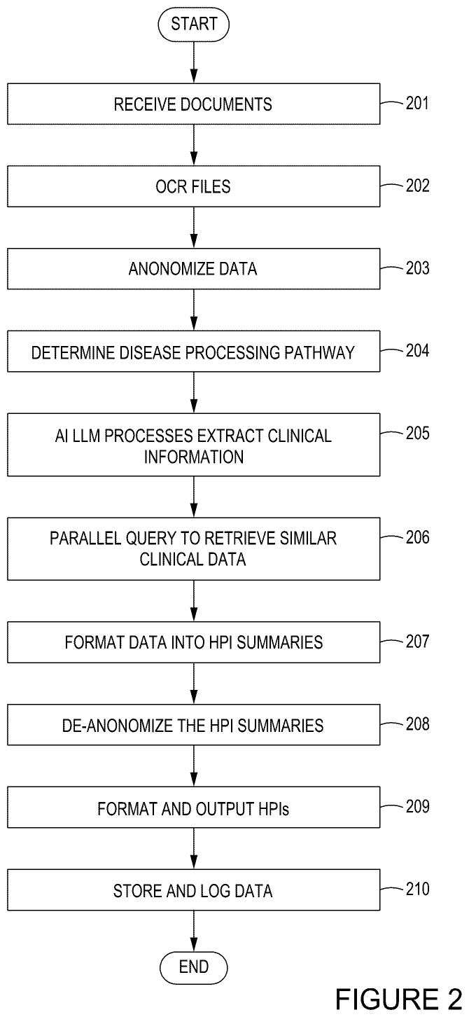

Referring to FIG. 2, the operation of the HPI Generator system 200 begins when a user initiates the process through a user interface device 201. This device allows a clinician or authorized user to upload patient-related documents or select existing files from an integrated electronic health record (EHR) system. In Step 201, these documents are received by the data ingestion module 102, which accepts a variety of file formats, including PDF, DOCX, scanned image files (e.g., TIFF, PNG, JPEG), HL7 messages, and FHIR-compliant JSON structures.

If any of the documents contain image-based or non-editable text, Step 202 involves sending those files to the OCR engine 103. The OCR engine performs optical character recognition using pre-processing techniques such as image enhancement, de-skewing, and segmentation to convert the embedded visual text into machine-readable content.

Following OCR, or directly from ingestion if the files were already in editable format, the system proceeds to Step 203, where the anonymization module 104 processes the document text. This module identifies protected health information (PHI) using rule-based parsing and natural language processing techniques such as named entity recognition. Sensitive items-such as names, dates of birth, addresses, and record numbers—are replaced with tokens or masked values, and the corresponding mapping is securely stored for re-identification at a later stage.

In Step 204, the system invokes the workflow engine 105 to determine the applicable disease-specific processing pathway. Based on user input, metadata, or content patterns, the workflow engine selects a predefined workflow such as for prostate, breast, or lung cancer, each of which contains tailored logic to identify, filter, and prioritize relevant document types and clinical elements.

Once the relevant documents are selected, Step 205 begins with the anonymized text being submitted to the AI processing module 106. This module includes a transformer-based large language model (LLM), either locally hosted or accessed via secure external endpoints (e.g., OpenAI or Microsoft Azure). The LLM is used to extract structured clinical information relevant to the history of present illness, such as symptom onset, duration, location, severity, associated signs, progression, and prior interventions.

To enhance the contextual accuracy of extraction, Step 206 involves a parallel query to the vector store and retrieval module 107. This module retrieves semantically similar cases, clinical notes, or pre-generated HPI templates stored as embeddings, and uses retrieval-augmented generation (RAG) to help the LLM compose higher-quality and contextually accurate outputs, especially in cases of sparse or noisy input data.

In Step 207, the extracted clinical data is transmitted to the HPI composition engine 108. This module formats the data into one or more candidate HPI summaries, which may be rendered as narrative paragraphs for readability or as structured, labeled data fields for use in clinical decision support tools. Each output version may be annotated with confidence scores, field-level validation statuses, or citations linking to source documents.

After HPI generation, the process moves to Step 208, where the de-anonymization module 109 uses the previously stored mapping table to re-insert original PHI into the summary. This enables the final HPI output to be patient-specific and suitable for storage or clinical use without compromising earlier data protection.

In Step 209, the completed and re-identified HPI summary is sent to the output formatter and export module 110. This module formats the output into the user's desired or system-compatible structure, which may include plain text, PDF, HL7 CDA documents, or FHIR JSON for direct import into an EHR system. The user may also view, edit, or approve the summary via the user interface device 101.

Finally, Step 210 involves recording all events, system outputs, user interactions, and intermediate results through the backend system controller 111. All data is securely stored within the data storage and audit log system 112, ensuring compliance with privacy regulations, enabling post hoc reviews, and supporting audit trails for safety and quality control. This end-to-end process enables the HPI Generator to automate the extraction, structuring, and generation of high-quality History of Present Illness summaries, thereby significantly reducing the documentation burden on clinicians while improving consistency, accuracy, and efficiency in clinical workflows-particularly in radiation oncology and other time-sensitive specialties.

The following detailed description is an example of a system, according to one example, that includes a specialized workflow designed for the processing of clinical data related to prostate cancer. This workflow is configured to identify and analyze the specific types of documents that are commonly used in the diagnosis, staging, and treatment planning of prostate cancer, particularly in the context of radiation oncology. Upon activation, the system begins by classifying and prioritizing incoming documents, either from an electronic health record or uploaded manually, that are typically associated with the clinical management of prostate cancer.

The workflow targets documents such as the urologist's consultation note, which often contains the initial clinical evaluation, presenting symptoms such as elevated PSA or urinary changes, the results of physical examinations including digital rectal exams, and initial impressions or differential diagnoses. The system extracts relevant clinical information from these notes, including the timing of symptoms, prior urologic history, and any immediate treatment recommendations or referrals.

It also reviews the most recent urology clinic note, which provides an updated assessment following diagnostic workup. This note often includes follow-up examination results, patient-reported symptom progression, recent PSA values, and additional imaging or pathology findings. The workflow compares the most recent note with prior entries to assess disease trajectory and identify whether the patient's condition is improving, worsening, or stable.

Another critical document evaluated by the system is the pathology report confirming malignancy, which typically results from a transrectal ultrasound-guided prostate biopsy. From this report, the system extracts structured diagnostic details such as Gleason score, ISUP grade group, number of cores positive, laterality, and perineural invasion status. These histopathologic indicators are essential for clinical staging and risk stratification.

The system further processes the prostate MRI report, which offers radiologic staging and lesion localization. From this report, key findings such as PIRADS scores, lesion size, presence of extracapsular extension, seminal vesicle invasion, and other relevant anatomical descriptors are extracted. The system is trained to distinguish between clinically significant findings and incidental or non-specific observations, ensuring only relevant content contributes to the HPI summary.

Laboratory reports containing prostate-specific antigen (PSA) test results are also reviewed. When multiple PSA readings are available, the system tracks trends over time, calculating derived metrics such as PSA velocity or doubling time if applicable. These quantitative trends play an important role in determining disease aggressiveness and urgency of intervention.

Patient intake forms and paperwork, including self-reported histories, are additionally parsed by the workflow. These documents may provide important context, such as family history of prostate cancer, prior surgical history, medication use, and lifestyle factors like smoking or alcohol use. The system analyzes both structured fields and free-text responses to compile a comprehensive view of the patient's health background.

Throughout this process, the system uses natural language processing and, where needed, optical character recognition to extract structured elements from free-text sources or scanned documents. Large language models assist in interpreting ambiguous language, summarizing findings, and cross-referencing data across multiple document types. A document prioritization mechanism ensures that the most current and relevant data are surfaced for inclusion in the final output.

The information extracted through this workflow is then passed to the HPI composition module, where it is synthesized into a cohesive summary. This summary includes the patient's presenting complaint, diagnostic timeline, key laboratory and imaging findings, and the clinical context necessary for staging and treatment planning. In some embodiments, the system may flag missing but expected data-such as a pending biopsy result or missing PSA test—and alert the user for manual review or completion.

By incorporating this prostate cancer-specific workflow, the system ensures that HPI summaries are not only automatically generated but also reflect the clinical depth, temporal structure, and specificity required by oncologists and multidisciplinary care teams managing prostate cancer. This targeted approach increases the reliability, utility, and clinical acceptance of the generated content in real-world practice

In one embodiment, clinicians will benefit from a simplified patient timeline that visualizes an essential, limited, selection of data as a “snapshot” of the patient's progress along a timeline plotting previous, current, and future known points of interest. Current systems are limited in scope due to program design and natural disparity of system types. In response, one embodiment of the present invention pertains to a system and methodology for generating one or more unified patient timeline views is described in detail herein. It includes the use of natural language processing (NLP) to interpret clinical orders and documentation to identify staging, history of previous illness, and diagnoses and large language model (LLM) algorithms to predict the relevant healthcare information, comorbidities, documentation, lab work, imaging, coding, and medications required for the oncology care team for population of a meaningful patient timeline for each disease presentation.

Electronic Health Record (EHR) systems and Oncology Information Systems (OIS) are essential for storing, maintaining, and presenting patient data to general care, specialty care, and Oncology clinicians. These systems house immense levels of data which are not integrated and are difficult to summarize or navigate. This system and methodology yield the clear visualization of a patient's oncology treatment in an efficient, streamlined, and interactive manner, increasing efficiency and effectiveness.

Simplified patient timeline that visualizes an essential, limited, selection of data as a “snapshot” of the patient's progress along a timeline plotting previous, current, and future known points of interest. A unified patient timeline view which visualizes a patient's oncology treatment in an efficient, streamlined, and interactive manner is generated and displayed. Data is culled from multiple customer systems including Ois, EHR (Electronic Health Record), and others. The timeline includes points where the patient has received a diagnosis, treatment plan, therapy, or otherwise had an encounter with a treatment facility or clinician. Each point is represented by a corresponding icon, label, and date. The points are interactive such that the user can view details in either a modal dialog box or an expanded detail area. For example, the user can click an icon labeled “patient diagnosis” to open details and see the patient's diagnosis code(s) and descriptions. The user can click a point labeled, “treatment plan” to view the document which outlines the patient's radiation therapy course of treatment. The user might click “Treatment Visit” to view on-site treatment details, such as fraction, modality, dosage, beam, and RT data. Other timeline points might include, “Dosimetry”, “Simulation”, “CT/PET/MRI scan”, “End of Treatment” or other relevant encounter/episode/occurrence information.

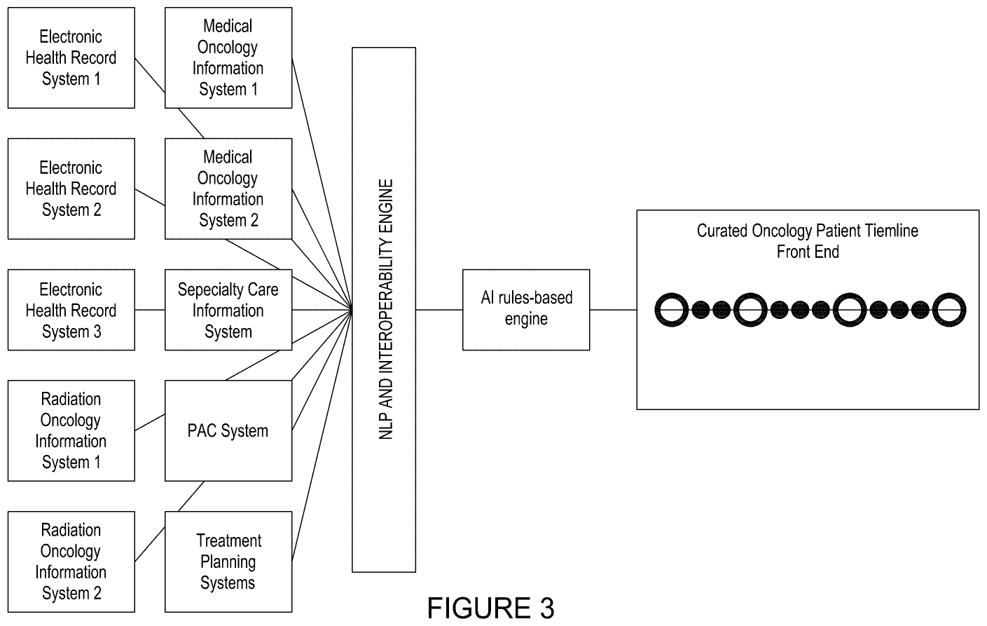

FIG. 3 shows a block diagram for a system and methodology for generating a unified patient timeline view. It illustrates an exemplary system architecture for generating a curated oncology patient timeline from heterogeneous clinical data sources. The system includes multiple upstream data sources, beginning with Electronic Health Record System 1 301, Electronic Health Record System 2 302, and Electronic Health Record System 3 303. Each of these systems may include unstructured and structured clinical data such as patient histories, lab reports, medications, visit summaries, and clinician notes.

In addition to general EHR systems, the system receives oncology-specific information from Medical Oncology Information System 1 304 and Medical Oncology Information System 2 305, which contain treatment regimens, infusion schedules, progress notes, and adverse event documentation. A Specialty Care Information System 306 aggregates data from subspecialties such as pathology, surgical oncology, and genetics, which may include tumor board notes, pathology reports, and genetic testing results.

Radiation oncology-specific information is provided by Radiation Oncology Information System 1 307 and Radiation Oncology Information System 2 308, each containing treatment plans, simulation records, daily treatment summaries, and dose calculations. Imaging data are accessed from PACS Systems 309, which provide both radiology reports and imaging metadata including modality, acquisition time, and anatomical focus. Additionally, Treatment Planning Systems 310 contribute information related to radiation dosimetry, including contours, treatment volumes, and dose-volume histograms.

All of the data from elements 301 through 310 are aggregated and interpreted through a Natural Language Processing (NLP) and Interoperability Engine 311. This component performs text extraction, clinical entity recognition, temporal analysis, and terminology mapping. It normalizes data into interoperable formats using ontologies such as SNOMED CT, LOINC, and FHIR profiles. The NLP engine identifies clinical concepts and their relationships, processes unstructured notes into structured fields, and determines event relevance and context.

The structured output from element 311 is then passed to an AI rules-based engine 312, which applies inference logic to identify, label, and sequence oncologic events. The engine includes condition-specific models that define the rules for how diagnosis, testing, treatment, response, and recurrence events should be interpreted and ordered. These models may incorporate temporal windows, severity thresholds, or combinations of clinical features to detect and connect relevant events.

The final output is displayed to the end-user via a Curated Oncology Patient Timeline Front End 313, which renders a graphical user interface showing a time-ordered series of key oncologic events. The visual timeline includes labeled nodes representing milestones such as presentation, biopsy, imaging, diagnosis, treatment initiation, follow-up, and recurrence. Each node may include tooltips, links to source documentation, or embedded metadata indicating event type, timestamp, and clinical significance.

This system architecture enables cross-system integration, AI-supported interpretation, and intuitive visualization of longitudinal oncology data, providing clinicians with a consolidated and clinically actionable view of a patient's cancer journey. The resulting display is a longitudinal patient chart. Initially, data is culled from multiple customer systems including OIS, EHR, and others. The unified patient timeline view which visualizes a patient's oncology treatment in an efficient, streamlined, and interactive manner will include points on the timeline where the patient has received a diagnosis, treatment plan, therapy, or otherwise had an encounter with a treatment facility or clinician. Essentially, the longitudinal timeline interface provides a visualization aggregate of data and documents from multiple disparate EHR systems, organizing them into a graphical format that displays key events like diagnoses, biopsies, surgeries, chemotherapy, and radiation therapy in a chronological sequence. The interface allows for intuitive interaction. Users can zoom into specific time periods or care events and view either summarized information or the original source documents. Additional features include layered views for different care teams (e.g., radiologists, surgical oncologists, nurses) and possibly node-based representations of cancer progression over time.

Various points can each be represented by a corresponding icon, label, and date. The points are interactive so the user can view details in either a modal dialog box or an expanded detail area. For example, the user can click an icon labeled “patient diagnosis” to open details and see the patient's diagnosis code(s) and descriptions. The user could click a point labeled, “treatment plan” to view the document which outlines the patient's radiation therapy course of treatment.

More examples include the user clicking on “Treatment visit” to view on-site treatment details like fraction, modality, dosage, beam, and RT data. Additional points might include “Dosimetry”, “Simulation”, “CT/PET/MRI scan”, “End of Treatment” or other relevant encounter, episode, or occurrence information. The timeline could also be filtered to show or highlight only “planning”, “treatment”, “support” “documents”, allowing for an overlap of multiple types where applicable. For example, a treatment visit might also include a generated document. It should be noted that the descriptions of the present system and methodology have been presented for purposes of illustration and description. They are not intended to be exhaustive or to limit the invention to the precise forms disclosed. The language and descriptions were chosen to best explain the principles of the present technology and its practical application.

The apparatus for generating a longitudinal patient timeline includes several interrelated modules and components designed to provide clinicians with a comprehensive, interactive visualization of a patient's historical medical data. At its core, the apparatus comprises a data ingestion module that receives a variety of clinical documents from one or more electronic health record systems. These documents may include radiology reports, pathology summaries, surgical notes, laboratory test results, progress notes, and specialist consultation records. The ingestion module is designed to handle differing formats and naming conventions across institutions, enabling robust interoperability.

A temporal extraction engine is responsible for parsing these documents to identify and extract time-stamped medical events. This engine uses natural language processing algorithms to detect both explicit date markers and implicit temporal references such as “two weeks ago” or “on postoperative day three.” It then normalizes these references into absolute calendar dates using the associated document creation timestamps and any referenced admission or encounter dates. By resolving ambiguities and aligning events to a unified timeline, the system provides accurate temporal coherence across disparate data sources.

To ensure consistency and reduce redundancy, a normalization module processes the extracted data by standardizing terminology, removing duplicates, and correlating events that may be referred to differently in separate documents. This module also retains provenance by associating each extracted event with its original document source, including metadata such as author, institution, and timestamp. This allows users to verify or audit any element displayed on the timeline.

The timeline generation engine operates using one or more processors and is stored in system memory. It constructs a chronological visual representation of the patient's medical history. Events are grouped by category, such as diagnosis, treatment, imaging, and lab work. The graphical timeline is rendered using a dedicated visualization engine that presents events as standardized visual markers. Distinct clinical pathways, such as those led by different care teams, are represented using color-coded swim lanes, allowing users to distinguish overlapping treatment streams across oncology, surgery, radiology, and primary care.

The apparatus also includes a user interface that presents the timeline to users in a navigable, interactive format. Users can zoom in to view events in detail or zoom out to see longitudinal patterns across months or years. Selecting an event on the timeline brings up associated source documents and the specific extracted data points derived from those documents. The interface is optimized for both desktop and mobile use and supports role-based views. Different care team members, such as oncologists, nurses, navigators, and administrative staff, are granted access to different subsets of data relevant to their clinical or operational roles.

To enhance utility during care coordination and follow-up planning, the system further comprises a visual annotation layer. This annotation layer allows users to add manual inputs directly onto the timeline interface. These may include written notes, flag markers indicating important events or abnormalities, and scheduled follow-up reminders. Each annotation is linked to specific events or time ranges on the timeline and may be color-coded or filtered according to user preferences or institutional protocols. These annotations can be shared across the care team, integrated with alerts or messaging systems, and stored in a secure audit trail to preserve clinical intent and decision-making history.

Together, these modules function as an intelligent and adaptive visualization platform that aggregates, standardizes, and renders a patient's historical medical data as a cohesive timeline. The system enables faster clinical understanding, improves documentation accuracy, and facilitates collaborative, longitudinal care planning across multidisciplinary healthcare teams.

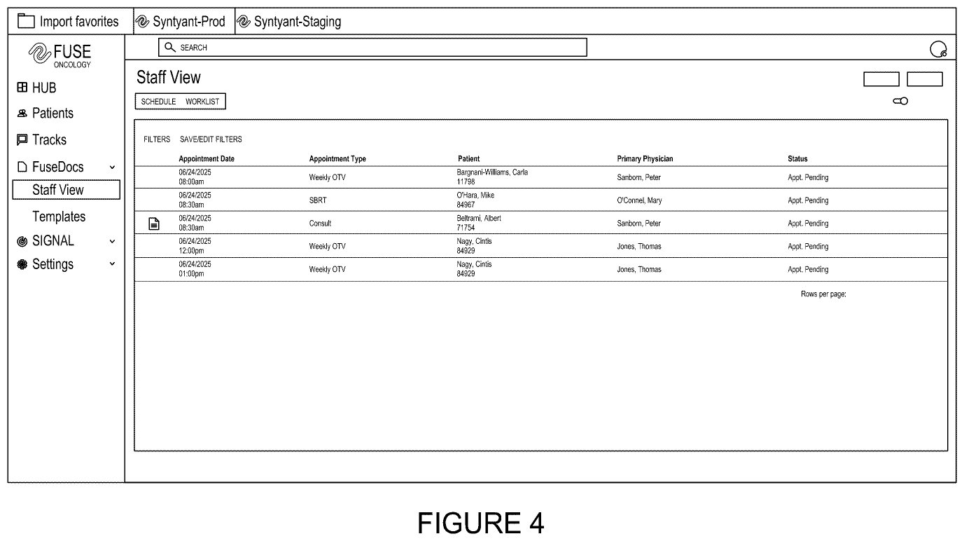

FIG. 4 shows an exemplary FuseDocs figure listing all scheduled notes. The relevant entry here is for the “Appointment Type: Consult,” where AI assists in generating its content. The screenshot displays the “Staff View” interface of one embodiment of the Oncology platform, which is designed to optimize oncology clinical workflows. This screenshot interface depicts the integration of intelligent scheduling and document automation into a unified, user-friendly environment for care teams. The “Staff View” screen is divided into columns showing appointment dates, types (e.g., Weekly OTV, SBRT, and Consult), patient identifiers, primary physician assignments, and appointment status. Notably, each row corresponds to an upcoming patient encounter, allowing staff to track clinical consults in real-time. The appointment status field, labeled “Appt. Pending,” signals that the automated document generation and data extraction processes tied to the HPI engine may be queued or in progress. Behind this interface, the system leverages dynamic document retrieval, AI-driven key data extraction, and disease-specific template generation, all configured to align with the context of each appointment type. For instance, a consult appointment would trigger a deeper search for diagnostic and staging data, while a Weekly OTV might prompt updates on lab work or treatment responses. By linking this scheduling interface to the underlying AI-driven HPI system, the invention enables real-time monitoring of clinical documentation readiness, prioritization of document workflows, and seamless physician validation, thereby reducing administrative burden and enhancing clinical decision support across the oncology care continuum.

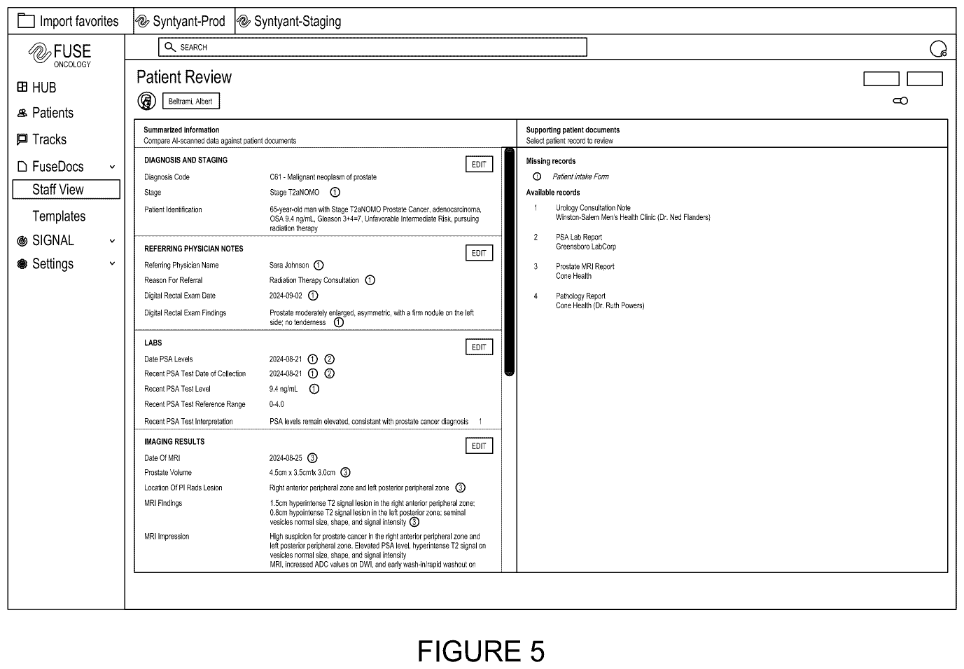

FIG. 5 shows a two-panel view after clicking on the consult note. The right side displays all documents retrieved from the system via dynamic document search, including alerts for any missing records. The left side organizes all extracted keys from the available records into broad sections-such as diagnosis and staging, referring physician notes, labs, imaging results, and pathology reports. These sections are dynamically configured by FuseDocs for enhanced interoperability across different systems and disease sites. Each key also shows which files the information was extracted from, combining counts if the same data appears in multiple documents. An Edit button allows users to modify or update values as needed.

At the top right, a “Verify Information” button moves to the next step, where AI populates multiple sections as described in the claims. If “Continue without Al” is selected, the HPI generator is bypassed and only static information retrieved from the OS is populated.

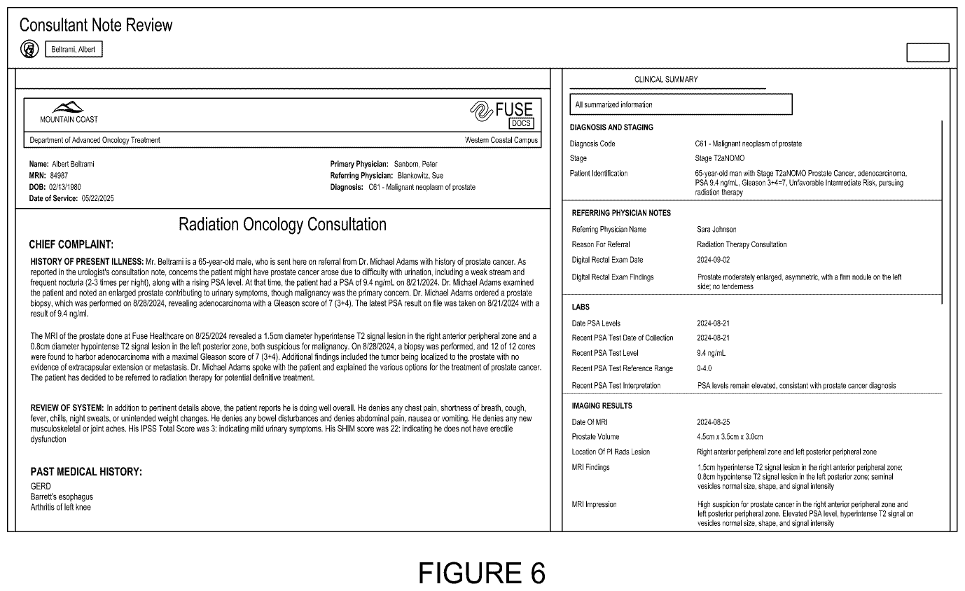

FIG. 6 shows a UI display window or panel after clicking the “Verify Information” button. The Radiation Oncology consultation not is automatically populated with the History of Present Illness and the Review of Systems, using the previously validated data.

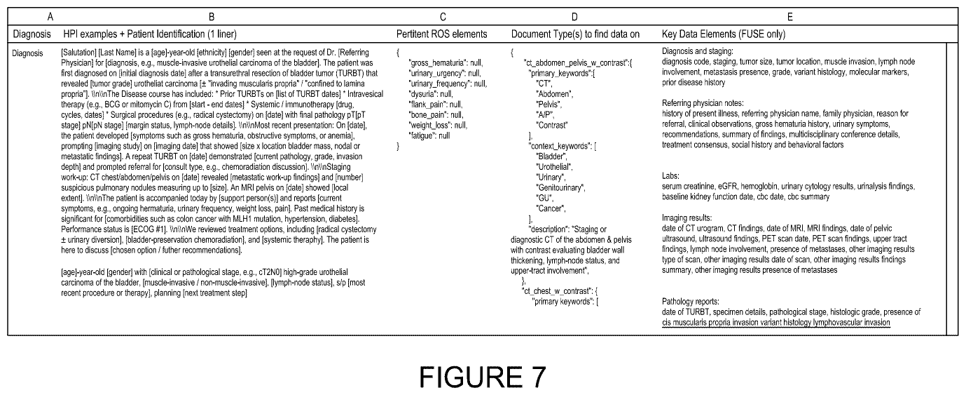

FIG. 7 shows a display of an example of the configurations that tracks each disease site. For example, column B contains the HPI template, which one can customize with placeholders and adjust to any desired tone. Column C lists the Review of Systems elements specific to the bladder disease site. Column D details the dynamic document search configuration, while column E outlines the key data elements that is extracted, categorized into the broader sections described above.

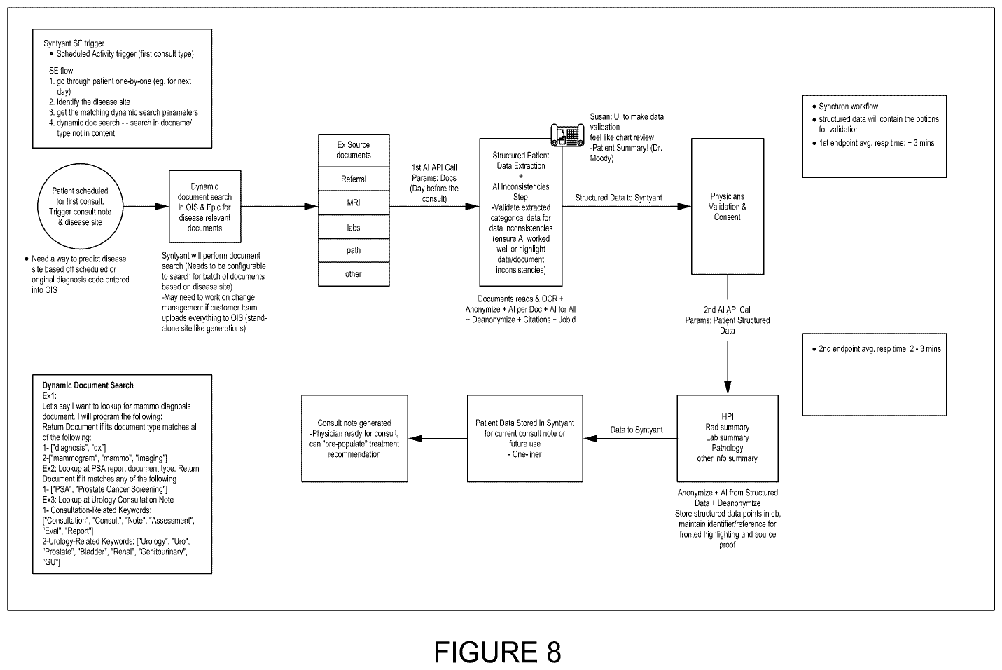

FIG. 8 shows an exemplary Consult Note AI workflow diagram. The diagram illustrates the end-to-end workflow of the HPI invention, highlighting the automation of consult preparation through intelligent document retrieval, AI-based data extraction, and structured physician validation. The process begins when a patient is scheduled for their first consult, triggering the system to identify the disease site and initiate a dynamic document search in the EHR for disease-relevant records such as referrals, imaging, labs, and pathology. These documents are then parsed using a specialized OCR engine and structured data extractor, which identifies key clinical values, resolves inconsistencies, and links each data point to its source for traceability. The structured data is sent to a secure server, where it becomes available for physician validation in a synchronous interface. After approval, the data is used to auto-generate the HPI and other consult sections (e.g., lab summary, pathology, and imaging) formatted in a disease-specific template. This output is anonymized, stored for future reuse, and supports downstream clinical documentation, treatment recommendations, and timeline visualization. The workflow dramatically reduces the time and cognitive burden on clinicians while ensuring accuracy, standardization, and interoperability in oncology documentation.

In conclusion, the disclosed invention offers a unique combination of disease-specific document retrieval, advanced OCR and data extraction, editable and verifiable structured information, and AI-powered generation of clinical notes. It intelligently adapts to oncologic contexts, ensuring both clinical accuracy and efficiency, and serving as a scalable solution capable of transforming documentation practices across a wide spectrum of cancer care settings.

What is claimed is:

1. A computer-implemented method executed on a system comprising one or more processors, a memory, and a display interface for generating a history of present illness summary, the method comprising:
receiving, via a document ingestion module, one or more medical documents containing unstructured or semi-structured clinical content in formats including scanned images, PDFs, and text-based reports;
converting non-text-based documents into machine-readable text using an optical character recognition engine integrated into the system;
processing the converted and original textual data using a trained natural language processing model configured to identify and extract clinical information including symptom descriptions, temporal references, anatomical locations, and progression markers;
applying a disease-specific processing workflow stored in a memory-accessible configuration file, the workflow comprising predefined clinical relevance rules and metadata filters based on patient specific information, to select and prioritize extracted clinical elements relevant to a history of present illness construction;
generating a structured narrative history of present illness summary comprising symptom onset, duration, location, and clinical progression; and
displaying the generated history of present invention summary on a user interface coupled to the system, wherein the history of present invention summary is optionally formatted for integration into an electronic health record system in a computer readable format.

2. The method of claim 1, wherein the medical documents include at least one of a urologist's note, pathology report, lab result, radiology report, or patient intake form.

3. The method of claim 1, further comprising retrieving additional supporting data from a clinical electronic health record system.

4. The method of claim 1, wherein the disease-specific workflow is selected automatically based on keyword recognition or patient metadata.

5. The method of claim 1, wherein the structured HPI output comprises a table of categorical elements including symptom location, severity, modifying factors, and associated findings.

6. The method of claim 1, further comprising generating confidence scores or flags for incomplete or ambiguous data fields.

7. The method of claim 1, wherein the HPI summary is generated in multiple formats for user selection.

8. A system for automated history of present illness (HPI) generation comprising:
a document ingestion module configured to receive clinical documents in multiple formats comprising PDF, DOCX, and image files;
an OCR engine configured to extract text from non-editable documents;
a preprocessing module configured to anonymize protected health information (PHI) prior to further processing;
a large language model configured to extract and synthesize clinical information from the documents based on a configurable disease-specific workflow; and
an output module configured to generate both narrative and structured HPI outputs suitable for physician review or EHR integration.

9. The system of claim 2, wherein the large language model is a fine-tuned transformer model trained on clinical data.

10. The system of claim 8, wherein the OCR engine uses image preprocessing techniques to enhance text recognition in scanned documents.

11. The system of claim 8, wherein the disease-specific workflow includes logic for parsing prostate cancer-specific inputs comprising PSA values and biopsy reports.

12. The system of claim 8, further comprising a user interface for real-time validation, editing, and correction of the generated HPI.

13. A non-transitory computer-readable medium storing instructions that, when executed by one or more processors, cause the system to:
receive and preprocess clinical documents containing patient information;
apply trained machine learning models to extract structured elements of a History of Present Illness (HPI) including chief complaint, associated symptoms, and clinical progression;
generate multiple suggested HPI summaries based on extracted data; and
present the summaries to a user with the option to select or edit the preferred version.

14. The non-transitory computer-readable medium of claim 13, wherein the instructions further cause the system to rank documents by relevance prior to processing.

15. A computer-implemented method for generating a History of Present Illness (HPI) summary, comprising:
receiving one or more medical documents comprising unstructured, semi-structured, or image-based clinical data;
performing a disease site-specific, dynamic document search to retrieve relevant documents from an electronic health record (EHR) system using a configurable list of keywords and document types associated with the identified disease;
processing documents individually using optical character recognition (OCR), where applicable, and natural language processing (NLP) to extract structured clinical information;
merging the extracted clinical information into a unified set of patient data keys, wherein each data key includes a citation linking to the source document;
generating a structured or narrative HPI summary using a disease-specific template based on the extracted keys; and
presenting the HPI summary in an interactive user interface for physician validation, editing, and final approval.

16. The method of claim 15, wherein the dynamic document search is updated based on national or global disease prevalence data to prioritize commonly encountered clinical documents.

17. The method of claim 15, wherein the disease-specific configuration file for document retrieval includes keywords, expected document types, and clinical markers for each cancer type.

18. The method of claim 15, wherein extracted clinical data includes a confidence score based on document quality, handwriting legibility, or value ambiguity.

19. The method of claim 15, wherein physician edits to extracted data trigger automatic regeneration of the HPI summary and all other related consult note sections.

20. The method of claim 15, wherein the user interface includes flagging for incomplete or conflicting data points and allows for structured feedback from the clinician.

21. The method of claim 15, wherein the generated summaries are accessible by other care team members, including nurses, care navigators, and the patient, in a controlled permission environment.

22. A system for oncology-specific clinical document extraction and consult note generation, comprising:
a document ingestion module configured to receive scanned images, PDFs, and EHR-native records;
a disease-informed search module configured to identify and prioritize relevant clinical documents based on disease site parameters;
an OCR engine fine-tuned for degraded or handwritten medical records, configured to extract clinical data from image-based inputs;
a machine learning model trained to extract patient data keys from individual documents and merge them with citation references;
a template engine configured to generate consult note sections, including HPI, pathology, lab work, radiology, and review of systems, based on the extracted data keys; and
a user interface for validation, editing, confidence scoring, and automatic regeneration of documentation.

23. The system of claim 22, wherein the disease-informed search module filters out irrelevant data, such as childhood injuries or non-cancer-related visits, based on context of the consult.

24. The system of claim 22, further comprising a visual dashboard for appointment tracking and consult readiness status based on document availability and AI-generated content progress.

25. The system of claim 22, wherein the template engine generates text using pre-defined disease-specific templates that include placeholders populated by validated data keys.

26. The system of claim 22, wherein the OCR engine is trained on samples of real-world low-resolution faxes and handwritten physician annotations, and incorporates preprocessing for enhanced legibility.

27. A computer-implemented method for generating a longitudinal patient timeline, comprising:
retrieving structured patient data and source documents from multiple EHR systems;
mapping diagnoses, procedures, lab results, imaging, and consults to a chronological graphical timeline;
displaying the timeline with interactive features including zoom, filter, and swim lane views for care teams or cancer progression stages; and
enabling access to original documents and citation-linked data via timeline nodes.

28. The method of claim 27, wherein the timeline allows swim lane visualization for multidisciplinary care teams including medical oncology, surgical oncology, and radiation oncology.

29. The method of claim 27, wherein the graphical timeline supports toggling between summary and full-detail views of diagnostic, therapeutic, and procedural milestones.

30. The method of claim 29, wherein the graphical timeline supports toggling between summary and full-detail views of diagnostic, therapeutic, and procedural milestones.

31. An apparatus for generating a longitudinal patient timeline from medical records, comprising:
a data ingestion module configured to receive a plurality of clinical documents from one or more electronic health record (EHR) systems comprising radiology reports, pathology summaries, surgical notes, lab results, and consultation records;
an extraction engine configured to extract time-stamped medical events from the clinical documents using natural language processing and metadata parsing;
a normalization module configured to standardize and correlate extracted events across varying document formats and naming conventions;
a timeline generation engine stored in memory and executed by one or more processors, the engine configured to construct a graphical timeline view of the patient's clinical history, wherein events are arranged chronologically and grouped by clinical category; and
a user interface configured to display the longitudinal timeline with interactive zoom functionality and selectable care-team swim lanes for diagnosis, treatment, imaging, and other healthcare events.

32. The apparatus of claim 31, wherein the temporal extraction engine is further configured to detect relative time references in narrative text, including phrases and calendar dates based on document timestamps.

33. The apparatus of claim 31, wherein the normalization module resolves duplicate or conflicting data entries by associating each event with its originating document and physician author.

34. The apparatus of claim 31 further comprising a document retrieval layer configured to query and rank relevant documents using a disease-specific search workflow, wherein relevance scores are assigned based on keyword frequency and document metadata.

35. The apparatus of claim 31, further comprising a role-based access control system that enables different views of the timeline for physicians, nurses, care navigators, and administrative users, each with access to different subsets of clinical data.

36. The apparatus of claim 31, wherein the user interface allows a user to select an event on the timeline and automatically display the source document and extracted key data points associated with that event.

37. The apparatus of claim 31 further comprising a visual annotation layer configured to allow users to add notes, flags, or follow-up reminders directly on the timeline, linked to specific events or time periods.
